(19) United States
(12) Patent Application Publication
(10) Pub. No.: US 2023/0391709 A1
(43) Pub. Date: Dec. 7, 2023

(54) METHOD FOR PREPARING ESTERIFIED ALKANE

(71) Applicant: Shanghai Jiao Tong University, Shanghai (CN)

(72) Inventors: Wenjun LU, Shanghai (CN); Lai XU, Shanghai (CN); Chong MEI, Shanghai (CN); Mengdi ZHAO, Shanghai (CN)

(73) Assignee: Shanghai Jiao Tong University, Shanghai (CN)

(21) Appl. No.: 18/451,108

(22) Filed: Aug. 17, 2023

Related U.S. Application Data
(63) Continuation of application No. PCT/CN2022/122863, filed on Sep. 29, 2022.

(30) Foreign Application Priority Data
Feb. 22, 2022 (CN) 202210162179.5

(51) Int. Cl. C07C 67/39; B01J 23/04

(52) U.S. Cl. CPC C07C 67/39; B01J 23/04

(57) ABSTRACT

The present disclosure relates to a method for preparing an esterified alkane. The method includes mixing a gaseous alkane with oxygen or air to obtain a mixed gas; adding a chlorine-containing catalyst and/or a nitrogen-containing catalyst to a light-transmission reaction vessel; then adding the mixed gas, an acid and a solvent in sequence to carry out a reaction under ambient pressure and an illumination condition; and then conducting analysis to obtain an NMR yield, followed by extraction, drying, filtration, distillation under reduced pressure and separation by column chromatography to obtain an esterified alkane. The present disclosure has the advantages that the reaction can be carried out under the conditions of ambient temperature and pressure with a cheap and safe chlorine-containing compound and a nitrogen-containing compound as a catalyst and air as an oxidant, and the method has energy-saving and economic effects, convenient and safe operation and environmental friendliness.

### CROSS-REFERENCE TO RELATED APPLICATION

This application is a continuation of international application of PCT application serial no. PCT/CN2022/122863, filed on Sep. 29, 2022, which claims the priority benefit of China application no. 202210162179.5, filed on Feb. 22, 2022. The entirety of each of the above-mentioned patent applications is hereby incorporated by reference herein and made a part of this specification.

### BACKGROUND OF THE INVENTION

### 1. Technical Field

The present disclosure relates to the technical field of chemical engineering, and in particular to a method for preparing an esterified alkane.

### 2. Background Art

Esterified alkanes are important organic chemicals, which are mainly used as solvents and intermediates in the synthesis of a variety of organic materials, drugs and daily necessities. During preparation of esters, corresponding alcohols of desired alkanes are usually obtained by biological fermentation, and then subjected to esterification with carboxylic acid to obtain corresponding esterified alkanes. The process has a long reaction route, a complicated product and a complex purification period. Moreover, food is consumed by the fermentation, and gases are released by metabolism of yeast, leading to food waste and environmental pollution. However, as important mineral resources, short chain alkanes represented by methane have extensive sources and abundant reserves, but are difficult to transport and convert. When the short chain alkanes are oxidized into active liquid products such as alcohols and esters, high economic and industrial significance is achieved. Since the alkanes have a high energy barrier in activation and products obtained by primary oxidation have a low energy barrier in further oxidation, a primary oxidation reaction of the alkanes is difficult to control, leading to easy over-oxidation of the alkanes. Due to such reason, the reaction has harsh oxidation conditions. At present, metal catalysts have been mainly used for oxidizing the methane under the conditions of high temperature and high pressure, where oxidants involved include K₂S₂O₈, H₂O₂, oxygen and the like.

As a part of air, the oxygen is a cheapest and most readily available oxidant. In 1990, according to a Moiseev research group, Co(III) trifluoroacetate was used as a catalyst in an oxidation reaction with oxygen at 180° C. to obtain methyl trifluoroacetate from trifluoroacetic acid. However, the yield was only four times of the use amount of the catalyst [Vargaftik, M. N.; Stolarov, I. P.; Moiseev, I. I. Journal of The Chemical Society, Chemical Communications 1990, (15), 1049-1050.].

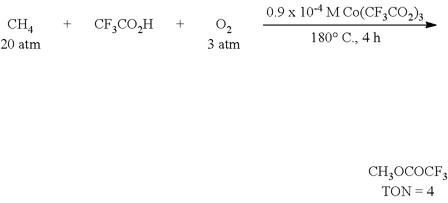

In 2013, a reaction for converting methane into methyl trifluoroacetate at 180° C. with a cobalt salt as a catalyst and oxygen as an oxidant was reported by Strassner et al. The conversion of methane in the reaction was as high as 50%, but the reaction had a poor inhibition effect on over-oxidation of the methane, so that a certain amount of the methane was over-oxidized into carbon dioxide and wasted [Strassner, T.; Ahrens, S.; Muehlhofer, M.; Munz, D.; Zeller, A. European Journal of Inorganic Chemistry 2013, 2013 (21), 3659-3663].

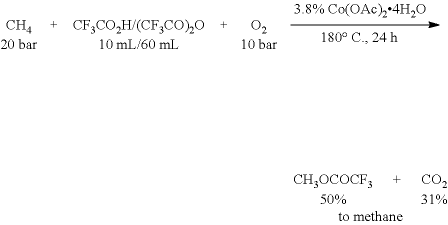

According to a report on the prior art published in 2006, palladium acetate was used as a main catalyst, sodium nitrite and p-benzoquinone were added, the p-benzoquinone was obtained by oxidizing p-phenol with oxygen, then electrons were transferred to an oxidation cycle of Pd(II) by the p-benzoquinone, and finally, a methyl trifluoroacetate product which was 7 times the dose of the catalyst was obtained at 80° C. [An, Z. A.; Pan, X.; Liu, X.; Han, X.; Bao, X. Journal of the American Chemical Society 2006, 128 (50), 16028-16029].

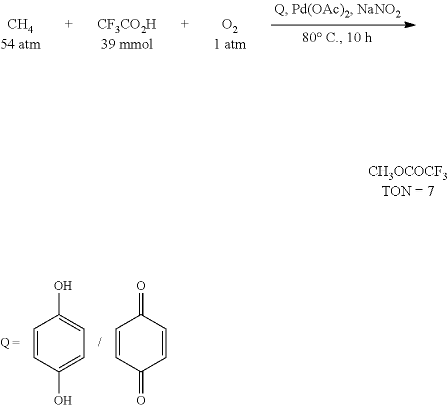

However, the converstion of methane in the reaction was still low, and heating and pressurization were required. Optimal oxidation conditions of the short chain alkanes, as a kind of flammable gas, include oxidizing the short chain alkanes with air under ambient temperature and pressure without using expensive metal catalysts and other materials as much as possible. Until now, reactions under such conditions have not been reported yet.

### SUMMARY OF THE INVENTION

Purposes of the present disclosure are to overcome the shortcomings of the prior art and provide a method for preparing an esterified alkane by using air or oxygen as an oxidant and a cheap chlorine compound and a nitrogen compound as a catalyst under an illumination condition. The method has the advantages of high conversion of reactants, mild reaction conditions, simple operation and environmental friendliness.

The purposes of the present disclosure can be realized by the following technical solutions.

A method for preparing an esterified alkane includes mixing a gaseous alkane with oxygen or air to obtain a mixed gas; adding a chlorine-containing catalyst and/or a nitrogen-containing catalyst to a light-transmission reaction vessel; then adding the mixed gas, an acid and a solvent in sequence to carry out a reaction under ambient pressure and an illumination condition; after the reaction is completed, adding CDCl₃ and an internal standard to a reaction mixture, and subjecting the mixture to drying, followed by sampling for ¹H NMR analysis to obtain an NMR yield; subjecting the mixture to extraction with CH₂Cl₂, followed by drying with anhydrous sodium sulfate, and filtration; and then subjecting a filtrate to distillation under reduced pressure to obtain a crude product, and subjecting the crude product to separation by column chromatography to obtain a pure esterified alkane product.

The reaction can be expressed by the following total reaction equation:

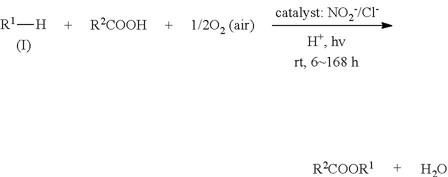

A chemical structure of the alkane is as shown in (I). R¹—H refers to all C₁-C₃₀ alkanes containing a sp³ C—H bond and simple substituted alkanes, and the gaseous alkane is preferably methane.

R² refers to a substituent group of organic carboxylic acid, preferably trifluoromethyl.

Further, the chlorine-containing catalyst is a chlorinated metal salt or hydrogen chloride.

Further, the nitrogen-containing catalyst includes nitric acid, nitrous acid, nitrate, nitrite and a nitrogen oxide.

Further, the acid includes all protonic acids, preferably hydrochloric acid or sulfuric acid, or no acid is additionally added.

Further, the molar amount of the chlorine-containing catalyst is 10% to 500% of that of the alkane; the molar amount of the nitrogen-containing compound is 0.01% to 100% of that of the alkane; and the molar amount of the acid is 100% to 500% of that of the chlorine-containing catalyst and/or the nitrogen-containing catalyst.

Further, the solvent is an acidic solvent or a neutral solvent, or no solvent is used. The acidic solvent is an organic acid, and the neutral solvent is CH₃NO₂, CH₂Cl₂ or CHCl₃.

Further, the illumination condition includes natural light or an external light source, and the external light source is an incandescent lamp, a linear fluorescent lamp, a compact fluorescent lamp, an LED lamp or an ultraviolet lamp with a power of greater than 4 W.

Further, the reaction is controlled at a temperature of −5° C. to 110° C. for 2-168 hours, and preferably, the reaction is conducted under a normal temperature condition for 24 hours.

Further, the esterified alkane prepared is a single ester substitute of the spa C—H bond of the alkane.

Compared with the prior art, the present disclosure has the following advantages.

(1) The esterified alkane, which usually needs to be obtained in multiple steps, is obtained in a milder and better selective way.

(2) According to a conventional esterification approach, methane is subjected to synthetic gas reforming with hydrogen to obtain methanol and then subjected to esterification. However, according to the method, methane is directly oxidized with air in one step, so that the method has a short synthetic route and simple and cheap raw materials, a lot of resources are saved, and energy consumption is reduced.

(3) A chlorine-containing reagent (such as NaCl) and a nitrogen-containing reagent (such as NaNO₂) which are convenient to store and transport are used as a catalyst. Compared with synthetic gases and other complex metal catalysts commonly used for preparation of methane, the catalyst has the advantages of being extremely low in price, easy to obtain, low in toxicity and convenient to treat.

(4) By using the air as an oxidant, the cost is greatly reduced, and the method is suitable for promotion to industrialization.

(5) Existing methods for oxidation of methane usually have the problem of over-oxidation, and a large amount of methane is over-oxidized into aldehydes, acids and even carbon dioxide, leading to waste of lots of raw materials. According to the method, carboxylic acid has a protective effect during oxidation of methane, and a primary oxidation product of the methane is obtained in a way with excellent selectivity and high yield. Either methyl ester is directly produced or the methane is further converted into methanol or other primary oxidation products of the methane, the method is better for oxidation of methane.

(6) Visible light (such as natural light) can be used as a light source in the reaction, and ultraviolet light and other special light sources are not required.

(7) The reaction can be carried out under ambient temperature and pressure conditions, dehydration of reaction reagents in advance is not required, and anhydrous protection of the reaction system during the reaction is also not required, so that the method has energy-saving and economic effects. In summary, the reaction has cheap and readily available raw materials, convenient and safe operation and environmental friendliness.

### DETAILED DESCRIPTION OF THE EMBODIMENTS

The present disclosure is described in detail with reference to the particular examples below. The following particular examples will be conducive to further understanding by those skilled in the art on the present disclosure, but are not intended to limit the present disclosure in any forms. It should be pointed out that various transformations and modifications can be made by those of ordinary skill in the art without departing from the spirit of the present disclosure, and all the transformations and modifications fall within the scope of protection of the present disclosure.

### Example 1

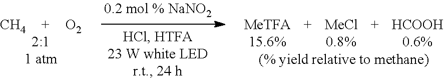

Methane was premixed with oxygen at a ratio of 2:1 to obtain a mixed gas. Sodium nitrite (0.0023 mmol) was added into a Schlenk tube, vacuumization was conducted for gas exchange, 35 mL of the mixed gas, 37% hydrochloric acid (1.91 mmol) and trifluoroacetic acid (0.7 mL) were added in sequence, then a screw opening was screwed, and stirring was conducted under illumination of a 23-watt LED white lamp 1 cm away from the reactor. After a reaction was completed, 10 mL of CDCl₃ was added and fully shaken, a quantitative internal standard (nitromethane) was added and stirred for 30 seconds, and 0.5 g of anhydrous sodium sulfate was added for precipitation for 10 minutes. Then, about 500 μL of a supernatant was taken and detected by ¹H NMR, and the yield was calculated.

This example illustrates a reaction at normal temperature, with the trifluoroacetic acid as a solvent, the methane and the oxygen as substrates/reactants, the nitrite as a catalyst whose dosage was 0.2% of that of the methane, and the hydrochloric acid as an acid under illumination of the 23-watt LED energy-saving lamp as a light source.

### Example 2

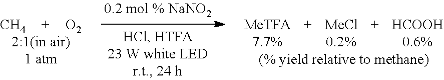

Methane was premixed with air at a ratio of 1:2.4 (where the ratio of the methane to oxygen was 2:1) to obtain a mixed gas. Sodium nitrite (0.0010 mmol) was added into a Schlenk tube, vacuumization was conducted for gas exchange, 35 mL of the mixed gas, 37% hydrochloric acid (0.85 mmol) and trifluoroacetic acid (0.3 mL) were added in sequence, then a screw opening was screwed, and stirring was conducted under illumination of a 23-watt LED white lamp 1 cm away from the reactor. After a reaction was completed, 10 mL of CDCl₃ was added and fully shaken, a quantitative internal standard (nitromethane) was added and stirred for 30 seconds, and g of anhydrous sodium sulfate was added for precipitation for 10 minutes. Then, about 500 μL of a supernatant was taken and detected by ¹H NMR, and the yield was calculated.

This example illustrates a reaction with the methane and the air as reactants.

### Example 3

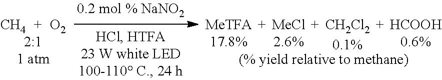

Methane was premixed with oxygen at a ratio of 2:1 to obtain a mixed gas. Sodium nitrite (0.0023 mmol) was added into a Schlenk tube, vacuumization was conducted for gas exchange, mL of the mixed gas, 37% hydrochloric acid (1.91 mmol) and trifluoroacetic acid (0.7 mL) were added in sequence, then a screw opening was screwed, stirring was conducted under illumination of a 23-watt LED white lamp 1 cm away from the reactor, and air-blowing was conducted with a heater 20 cm away from the reactor. After a reaction was completed, cooling was conducted to normal temperature, 10 mL of CDCl₃ was added and fully shaken, a quantitative internal standard (nitromethane) was added and stirred for 30 seconds, and 0.5 g of anhydrous sodium sulfate was added for precipitation for 10 minutes. Then, about 500 μL of a supernatant was taken and detected by ¹H NMR, and the yield was calculated.

This example illustrates a reaction at a heating temperature of 110° C.

### Example 4

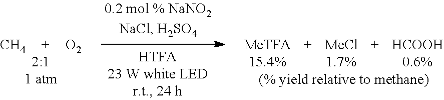

Methane was premixed with oxygen at a ratio of 2:1 to obtain a mixed gas. Sodium chloride (0.95 mmol) and sodium nitrite (0.0023 mmol) were added into a Schlenk tube in sequence, vacuumization was conducted for gas exchange, 35 mL of the mixed gas, 10 M sulfuric acid (1.91 mmol) and trifluoroacetic acid (0.7 mL) were added in sequence, then a screw opening was screwed, and stirring was conducted under illumination of a 23-watt LED white lamp 1 cm away from the reactor. After a reaction was completed, 10 mL of CDCl₃ was added and fully shaken, a quantitative internal standard (nitromethane) was added and stirred for 30 seconds, and 0.5 g of anhydrous sodium sulfate was added for precipitation for 10 minutes. Then, about 500 μL of a supernatant was taken and detected by ¹H NMR, and the yield was calculated.

This example illustrates a reaction when the chlorine-containing compound is sodium chloride and the acid is sulfuric acid.

### Example 5

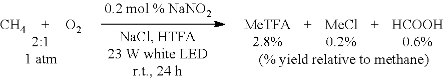

Methane was premixed with oxygen at a ratio of 2:1 to obtain a mixed gas. Sodium chloride (0.95 mmol) and sodium nitrite (0.0023 mmol) were added into a Schlenk tube in sequence, vacuumization was conducted for gas exchange, 35 mL of the mixed gas and trifluoroacetic acid (0.7 mL) were added in sequence, then a screw opening was screwed, and stirring was conducted under illumination of a 23-watt LED white lamp 1 cm away from the reactor. After a reaction was completed, 10 mL of CDCl₃ was added and fully shaken, a quantitative internal standard (nitromethane) was added and stirred for 30 seconds, and 0.5 g of anhydrous sodium sulfate was added for precipitation for 10 minutes. Then, about 500 μL of a supernatant was taken and detected by ¹H NMR, and the yield was calculated.

This example illustrates a reaction when the chlorine-containing compound is sodium chloride, the acid is trifluoroacetic acid and no solvent is used, or a reaction when the chlorine-containing compound is sodium chloride, the solvent is trifluoroacetic acid and no additional acid is used.

### Example 6

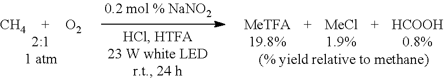

Methane was premixed with oxygen at a ratio of 2:1 to obtain a mixed gas. Sodium nitrate (0.0023 mmol) was added into a Schlenk tube, vacuumization was conducted for gas exchange, 35 mL of the mixed gas, 37% hydrochloric acid (1.91 mmol) and trifluoroacetic acid (0.7 mL) were added in sequence, then a screw opening was screwed, and stirring was conducted under illumination of a 23-watt LED white lamp 1 cm away from the reactor. After a reaction was completed, 10 mL of CDCl₃ was added and fully shaken, a quantitative internal standard (nitromethane) was added and stirred for 30 seconds, and 0.5 g of anhydrous sodium sulfate was added for precipitation for 10 minutes. Then, about 500 μL of a supernatant was taken and detected by ¹H NMR, and the yield was calculated.

This example illustrates a reaction when the nitrogen-containing compound is nitrate.

### Example 7

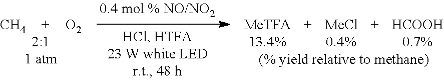

Methane was premixed with oxygen at a ratio of 2:1 to obtain a mixed gas. Vacuumization was conducted on a Schlenk tube for gas exchange, 35 mL of the mixed gas and a preprepared nitrogen oxide gas (0.0045 mmol), 37% hydrochloric acid (1.91 mmol) and trifluoroacetic acid (0.7 mL) were added in sequence, then a screw opening was screwed, and stirring was conducted under illumination of a 23-watt LED white lamp 1 cm away from the reactor. After a reaction was completed, 10 mL of CDCl₃ was added and fully shaken, a quantitative internal standard (nitromethane) was added and stirred for 30 seconds, and 0.5 g of anhydrous sodium sulfate was added for precipitation for 10 minutes. Then, about 500 μL of a supernatant was taken and detected by ¹H NMR, and the yield was calculated.

This example illustrates a reaction when the nitrogen-containing compound is a nitrogen oxide.

### Example 8

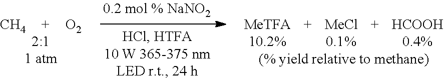

Methane was premixed with oxygen at a ratio of 2:1 to obtain a mixed gas. Sodium nitrite (0.0023 mmol) was added into a Schlenk tube, vacuumization was conducted for gas exchange, 35 mL of the mixed gas, 37% hydrochloric acid (1.91 mmol) and trifluoroacetic acid (0.7 mL) were added in sequence, then a screw opening was screwed, and stirring was conducted under illumination of a 10-watt 365-375 nm LED ultraviolet lamp 1 cm away from the reactor. After a reaction was completed, 10 mL of CDCl₃ was added and fully shaken, a quantitative internal standard (nitromethane) was added and stirred for 30 seconds, and 0.5 g of anhydrous sodium sulfate was added for precipitation for 10 minutes. Then, about 500 μL of a supernatant was taken and detected by ¹H NMR, and the yield was calculated.

This example illustrates a reaction under illumination of the 10-watt LED ultraviolet lamp as a light source.

### Example 9

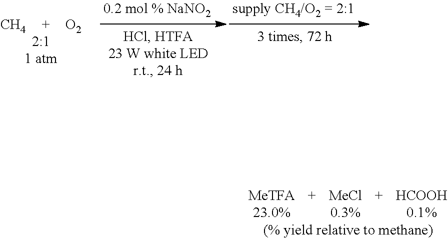

Methane was premixed with oxygen at a ratio of 2:1 to obtain a mixed gas. Sodium nitrite (0.0045 mmol) was added into a quartz sheet, vacuumization was conducted for gas exchange, 50 mL of the mixed gas, 37% hydrochloric acid (4.40 mmol) and trifluoroacetic acid (1.6 mL) were added in sequence, then a valve was turned off, and stirring was conducted under illumination of a 23-watt LED white lamp 1 cm away from the reactor. 24 hours later, 10 mL of the mixed gas was supplemented. 24 hours later, 10 mL of the mixed gas was supplemented. 24 hours later, 10 mL of the mixed gas was supplemented. After a reaction was completed, 10 mL of CDCl₃ was added and fully shaken, a quantitative internal standard (nitromethane) was added and stirred for 30 seconds, and 0.5 g of anhydrous sodium sulfate was added for precipitation for 10 minutes. Then, about 500 μL of a supernatant was taken and detected by ¹H NMR, and the yield was calculated.

This example illustrates a reaction when a material is continuously supplemented.

### Example 10

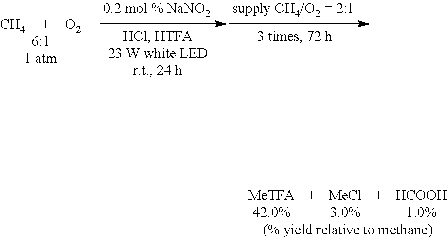

Methane was premixed with oxygen at a ratio of 6:1 to obtain a mixed gas. Sodium nitrite (0.0045 mmol) was added into a quartz sheet, vacuumization was conducted for gas exchange, 50 mL of the mixed gas, 37% hydrochloric acid (4.40 mmol) and trifluoroacetic acid (1.6 mL) were added in sequence, then a valve was turned off, and stirring was conducted under illumination of a 23-watt LED white lamp 1 cm away from the reactor. 24 hours later, 10 mL of the mixed gas was supplemented. 24 hours later, 10 mL of the mixed gas was supplemented. 24 hours later, 10 mL of a mixed gas of methane and oxygen at a ratio of 2:1 was supplemented. After a reaction was completed, 10 mL of CDCl₃ was added and fully shaken, a quantitative internal standard (nitromethane) was added and stirred for 30 seconds, and 0.5 g of anhydrous sodium sulfate was added for precipitation for 10 minutes. Then, about 500 μL of a supernatant was taken and detected by ¹H NMR, and the yield was calculated.

This example illustrates a reaction when the methane and the oxygen are used as initial raw materials at a ratio of 6:1 and then supplemented continuously.

### Example 11

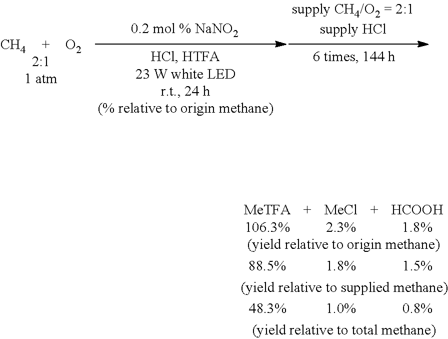

Methane was premixed with oxygen at a ratio of 2:1 to obtain a mixed gas. Sodium nitrite (0.0023 mmol) was added into a quartz sheet, vacuumization was conducted for gas exchange, 35 mL of the mixed gas, 37% hydrochloric acid (1.91 mmol) and trifluoroacetic acid (0.7 mL) were added in sequence, then a valve was turned off, and stirring was conducted under illumination of a 23-watt LED white lamp 1 cm away from the reactor. 24 hours later, 7 mL of the mixed gas and the 37% hydrochloric acid (1.91 mmol) were supplemented for six times repeatedly. After a reaction was completed, 10 mL of CDCl₃ was added and fully shaken, a quantitative internal standard (nitromethane) was added and stirred for 30 seconds, and 0.5 g of anhydrous sodium sulfate was added for precipitation for 10 minutes. Then, about 500 μL of a supernatant was taken and detected by ¹H NMR, and the yield was calculated.

This example illustrates a reaction when materials are continuously supplemented for a long time.

What is claimed is:

1. A method for preparing an esterified alkane, comprising mixing a gaseous alkane with oxygen or air to obtain a mixed gas; adding a chlorine-containing catalyst and/or a nitrogen-containing catalyst to a light-transmission reaction vessel; adding the mixed gas, an acid and a solvent in sequence to carry out a reaction under normal pressure and an illumination condition; and conducting analysis to obtain a nuclear magnetic yield, followed by extraction, drying, filtration, distillation under reduced pressure and separation by column chromatography to obtain an esterified alkane.

2. The method for preparing an esterified alkane according to claim 1, wherein the gaseous alkane is methane.

3. The method for preparing an esterified alkane according to claim 2, wherein a volume ratio of the gaseous alkane to oxygen or air is (2-6):1.

4. The method for preparing an esterified alkane according to claim 1, wherein the chlorine-containing catalyst is a chlorinated metal salt or hydrogen chloride.

5. The method for preparing an esterified alkane according to claim 1, wherein the nitrogen-containing catalyst comprises nitric acid, nitrous acid, nitrate, nitrite and a nitrogen oxide.

6. The method for preparing an esterified alkane according to claim 1, wherein the acid is a protonic acid.

7. The method for preparing an esterified alkane according to claim 1, wherein a molar amount of the chlorine-containing catalyst is 10% to 500% of that of the alkane; a molar amount of the nitrogen-containing compound is 0.01% to 100% of that of the alkane; and a molar amount of the acid is 100% to 500% of that of the chlorine-containing catalyst and/or the nitrogen-containing catalyst.

8. The method for preparing an esterified alkane according to claim 1, wherein the solvent is an acidic solvent or a neutral solvent, the acidic solvent is an organic acid, and the neutral solvent is CH₃NO₂, CH₂Cl₂ or CHCl₃.

9. The method for preparing an esterified alkane according to claim 1, wherein the illumination condition comprises natural light or an external light source, and the external light source is an incandescent lamp, a linear fluorescent lamp, a compact fluorescent lamp, an LED lamp or an ultraviolet lamp with a power of greater than 4 W.

10. The method for preparing an esterified alkane according to claim 1, wherein the reaction is controlled at a temperature of −5° C. to 110° C. for 2-168 hours.
